(19) United States
(12) Patent Application Publication
(10) Pub. No.: US 2005/0153392 A1
(43) Pub. Date: Jul. 14, 2005

(54) Transgenesis with humanized immunoglobulin loci

(76) Inventors: Roland Buelow, Palo Alto, CA (US); Wim van Schooten, Sunnyvale, CA (US)

Correspondence Address: HELLER EHRMAN LLP, 275 MIDDLEFIELD ROAD, MENLO PARK, CA, 94025-3506, US

(21) Appl. No.: 10/916,082

(22) Filed: Aug. 10, 2004

Related U.S. Application Data
(60) Provisional application No. 60/494,390, filed on Aug. 11, 2003.

(51) Int. Cl. C12P 21/02; C07H 21/04; C12N 5/06; C07K 16/44

(52) U.S. Cl. 435/69.1; 435/320.1; 435/328; 435/455; 530/388.15; 536/23.53

(57) ABSTRACT

The invention concerns recombinase-mediated transfer of human or humanized immunoglobulin loci into the genome of non-human transgenic animals. In particular, the invention relates to improved methods to integrate human and/or humanized immunoglobulin loci into the genome of non-human animals using transgenic constructs which contain immunoglobulin loci comprising human gene sequences and a site recognized by a site specific recombinase which can be used to catalyze the insertion of the transgene into the animal's genome.

### CROSS-REFERENCE TO RELATED APPLICATIONS

This is a non-provisional appliction filed under 37 C.F.R. 1.53(b), claiming priority under U.S.C. Section 119(e) to Provisional Application No. 60/494,390 filed Aug. 11, 2003.

### BACKGROUND OF THE INVENTION

### FIELD OF THE INVENTION

The present invention concerns methods and means to produce humanized antibodies from transgenic non-human animals. The invention specifically relates to improved methods to integrate human and/or humanized immunoglobulin loci into the genome of non-human animals. The transgenic vectors contain immunoglobulin loci comprising human gene sequences, which are capable of undergoing gene rearrangement and/or gene conversion and/or hypermutation in transgenic non-human animals to produce diversified humanized antibodies. In addition, the transgenic constructs contain a site recognized by a site specific recombinase which can be used to catalyze the insertion of the transgene into the animal's genome. Transgenic animals with transgenes integrated by a recombinase express higher levels of humanized antibodies compared to transgenic animals with randomly integrated transgenes. The humanized antibodies obtained have minimal immunogenicity to humans and are appropriate for use in the therapeutic treatment of human subjects.

Antibodies are an important class of pharmaceutical products that have been successfully used in the treatment of various human diseases and conditions, such as infectious diseases, cancer, allergic diseases, prevention of transplant rejection and graft-versus-host disease.

Recombinant immunoglobulin loci encoding human or humanized antibodies have been developed. Various methods to insert DNA sequences in the genome of animals have been described and integration of human or humanized immunoglobulin loci into the genome of animals was shown to result in the production of human antibodies. See, e.g., Jakobovits et al., Proc. Natl. Acad. Sci USA, 90: 2551 (1993); Jakobovits et al., Nature, 362: 255 (1993); Bruggemann et al., Year in Immunol., 7: 33 (1993), Kuroiva et al., Nature Biotech 20:889 (2002). Unfortunately all current transformation procedures of eukaryotic cells result in a very low integration frequency of DNA into the genome of cells (including oocytes, spermatozoa, zygotes, spermatogonia, blsastomers, stem cells, etc.) and, therefore, the frequency of transgenic founder animals is low. In addition, the random integration of introduced DNA into the genome of a host cells can be lethal. Lastly, transgene expression is dependent on the integration site and a large number of founder animals have to be screened for the identification of animals expressing sufficient amounts of human(ized) antibodies.

Therefore, for the expression of heterologous immunoglobulin genes in animals it is desirable to have a method for the efficient integration of human or humanized immunoglobulin loci in the genome of non-human animals that results in a high frequency of founder animals expressing high amounts of antibodies with no or minimal immunogenicity to humans.

### SUMMARY OF THE INVENTION

In one aspect, the present invention concerns an isolated nucleic acid molecule comprising one or more human(ized) immunoglobulin loci and a nucleotide sequence recognized specifically by a recombinase. The nucleic acid may be part of a targeting construct or expression cassette, which may be circular (for any type of recombinase) or linear (for transposases).

In another aspect, the invention concerns a method of site-specifically introducing an isolated nucleic acid molecule comprising one or more human(ized) immunoglobulin loci into the genome of a cell, in particular a eukaryotic host cell. The method comprises introducing (i) a targeting construct comprising one or more human(ized) immunoglobulin loci and a first recombination site, and (ii) a site specific recombinase into the eukaryotic cell, wherein the genome of the cell comprises a second recombination site native to or introduced into the genome of the cell, and wherein recombination between the first and the second recombination sites is facilitated by the site-specific recombinase. The cell is maintained under conditions that allow recombination between first and second recombination sites and the recombination is mediated by the site-specific recombinase. The result of the recombination is site-specific integration of one or several human(ized) immunoglobulin loci in the genome of the eukaryotic cell. The recombinase may be introduced into the cell before, concurrently with, or after introducing the circular construct comprising one or several human(ized) immunoglobulin loci. The recombinase may be introduced as an enzymatically active protein. Alternatively, the expression of recombinase may be accomplished through introduction of messenger RNA encoding recombinase. Yet another way of introducing the recombinase is through introduction of a recombinant expression plasmid encoding recombinase.

In a certain embodiment, the recombinase may be a site-specific recombinase encoded by a phage selected from the group consisting of φC31, Temperate Lactococcal Bacteriophage TP901-1, and R4. The recombinase may catalyze recombination between a bacterial genomic recombination site (attB) and a phage genomic recombination site (attP), or the first site may comprises a pseudo-attB site and/or the second site may comprises a pseudo-attP site, or vice-versa. For the integration of the construct comprising one or several human(ized) immunoglobulin loci into the genome of a cell one of the recognition sites has to be located in the cellular DNA.

In another embodiment, the recombinase may facilitate recombination between an attB (pseudo)site and an attP (pseudo)site, wherein the recombinase mediates production of recombination sites that are no longer substrates for the recombinase.

In additional embodiments, the recombinase may facilitate recombination between two recombinase specific recognition sites, such as loxP or FRT sites. In this embodiment the recombinase may be Cre and FLP and the like. In case such recombination specific recognition sequence is not present in the cellular genome, it has to be introduced before integration of the construct comprising one or several human(ized) immunoglobulin loci. Recombinase specific recognition sequences can be introduced into the cellular genome using standard procedure including viral and non-viral vectors. Preferably, one or several such sites are introduced into the cellular genome in combination with a marker gene that allows analysis of gene expression levels at the integration site. Suitable marker genes include, without limitation, luciferase, Green-Fluorescence-Protein, Chloramphenicol-Acetyl-Transferase, and the like. Subsequent to the isolation of a cell comprising a recombinase specific recognition sequence at a site that allows high gene expression, such cell can be used for the integration of a construct comprising one or several human(ized) immunoglobulin loci.

In a further embodiment, the invention is used for the generation of transgenic animals wherein the integration of the transgenic construct comprising one or several human(ized) immunoglobulin loci was facilitated by a recombinase.

In a further embodiment, the invention is used for the generation of non-immunogenic antibodies using transgenic animals wherein the integration of the transgenic construct comprising one or more human(ized) immunoglobulin loci was facilitated by a recombinase.

### BRIEF DESCRIPTION OF THE DRAWINGS

The present invention concerns methods and means to produce humanized fragments (Unit1-4) consisting of human VH3 gene fragments and rabbit spacer and intron sequences were combined with parts of BAC 219D23, 27N5 and Fos15B containing human Cμ, Cγ and J_{H}.

FIG. 2 shows a humanized light chain locus. Two synthetic DNA fragments containing human V pseudogenes and chicken spacer sequences were combined with a fragment derived from BAC 179L1 containing human Ck and rabbit intron and spacer sequences.

FIG. 3 is a schematic depiction of a construct containing a humanized light chain immunoglobulin locus and an attB site. The chicken light chain locus was modified through replacement of chicken Cλ with human Cλ. A synthetic human VλJλ was inserted into the chicken J locus. A 35 kb fragment encoding the entire modified locus was cloned into pGEM13Zf(+) and an attB site was inserted using synthetic oligonucleotides.

FIG. 4 shows a DNA sequence (SEQ ID NO 27) encoding C31 integrase.

FIG. 5 illustrates CRE mediated cassette exchange and integration.

### DETAILED DESCRIPTION OF THE PREFERRED EMBODIMENTS

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “non-human animal” as used herein includes, but is not limited to, mammals such as, for example, non-human primates, rodents (e.g. mice and rats), and non-rodent animals, such as, for example, rabbits, pigs, sheep, goats, cows, pigs, horses and donkeys. It also includes birds (e.g., chickens, turkeys, ducks, geese and the like). The term “non-primate animal” as used herein refers to mammals other than primates, including but not limited to the mammals specifically listed above.

The term “transgenic (non-human) animal” refers to any (non-human) animal in which one or more cells contain heterologous nucleic acid introduced by human intervention.

The term “transgene” as used herein refers to a polynucleotide introduced into a cell by human intervention.

The term “transgene construct” is used herein to refer to a polynucleotide molecule, which contains a structural gene of interest and other sequences facilitating gene transfer. The transgene construct can, for example, be a vector (plasmid) comprising the gene of interest operatively linked to regulatory sequences.

The terms “polynucleotide” and “nucleic acid” are used interchangeably, and, when used in singular or plural, generally refer to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The terms “recombinase” and “site-specific recombinase” are used interchangeably and in the broadest sense, and refer to a group of enzymes that can facilitate site specific recombination between defined recombinase specific recognition sequences (recombination sites), where the sites are physically separated on a single nucleic acid molecule or where the sites reside on separate nucleic acid molecules. The sequences of the defined recombination sites are not necessarily identical. Within this group are several subfamilies including “integrases” (for example, site-specific recombinases, like Cre, Cre-like, FLP and λ integrase) and “resolvases/invertases” (for example, φC31 integrase, R4 integrase, and TP-901 integrase). In addition, the term “recombinase” specifically includes transposases and retrotransposases like Drosophila mariner, sleeping beauty transposase, L1, Tol2 Tc1, Tc3, Mariner (Himar 1), M, and the like.

The term “wild-type recombination site” as used herein refers to a recombination site normally used by a recombinase such as, for example, an integrase or transposase.

By “pseudo-recombination site” means a site at which recombinase can facilitate recombination even though the site may not have a sequence identical to the sequence of its wild-type recombination site.

The term “recombinase-mediated integration of a transgene” is used to refer to integration mediated by an encoded and expressed recombinase, that facilitates specific integration of the transgene into the genome of a cell rather than random integration and thus results in a higher percentage of transgenic cells.

The term “expression cassette encoding recombinase” refers to sequences comprising the recombinase gene of interest operatively linked to a suitable promoter and/or regulatory sequences that promote recombinase gene expression.

The term “retroviral vector” is used to refer to a retrovirus or retroviral particle, which is capable of entering a cell and integrating the retroviral genome into the genome of the host cell.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lyrnph system and at increased levels by myelomas.

“Native antibodies and immunoglobulins” are usually heterotetrameric glycoproteins of about 150,000 daltons, composed of two identical light (L) chains and two identical heavy (H) chains. Each light chain is linked to a heavy chain by covalent disulfide bond(s), while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains, Chothia et al., J. Mol. Biol. 186:651 (1985); Novotny and Haber, Proc. Natl. Acad. Sci. U.S.A. 82:4592 (1985).

The terms “antibody diversity” and “antibody repertoire” are used interchangeably, and refer to the total of all antibody specificities that an organism is capable of expressing.

An Ig locus having the capacity to undergo gene rearrangement and/or gene conversion is also referred to herein as a “functional” Ig locus, and the antibodies with a diversity generated by a functional Ig locus are also referred to herein as “functional” antibodies or a “functional” repertoire of antibodies.

The term “monoclonal antibody” is used to refer to an antibody molecule synthesized by a single clone of B cells.

The term “polyclonal antibody” is used to refer to a population of antibody molecules synthesized by a population of B cells.

B. Detailed Description

The present invention provides an efficient method for introducing one or more human or humanized immunoglobulin loci into the genome of a recipient non-human animal by recombinase-mediated site-specific integration.

In particular, the invention concerns a method for the introduction of an expression cassette, comprising one or more human or humanized immunoglobulin loci and a first recombination site, into the genome of a cell of a non-human animal. The genome of the recipient cell comprises a second recombination site, which may be native to the cell or may have been introduced into the cell. Recombination between the first and the second recombination sites is facilitated by an appropriate site-specific recombinase, which recognizes the first and second recombination sites. As a result, instead of random integration, the one or more human or humanized immunoglobulin loci is/are integrated in the genome of the recipient cell in a site-specific manner, which greatly improves integration efficiency.

Methods for introducing human or humanized immunoglobulin loci into the genome of non-human animals are known in the art. Thus, the introduction of human immunoglobulin genes into the genome of mice resulted in expression of a diversified human antibody repertoire in genetically engineered mice. Jakobovits et al., Proc. Natl. Acad. Sci USA, 90: 2551 (1993); Jakobovits et al., Nature, 362: 255 (1993); Bruggemann et al., Year in Immunol., 7: 33 (1993), Kuroiva et al., Nature Biotech 20:889 (2002). The production of humanized antibodies in transgenic non-human animals is described in PCT Publication No. WO 02/12437, published on Feb. 14, 2002, the disclosure of which is hereby expressly incorporated by reference in its entirety. WO 02/12437 describes genetically engineered non-human animals containing one or more humanized immunoglobulin loci which are capable of undergoing gene rearrangement and gene conversion in transgenic non-human animals, including animals in which antibody diversity is primarily generated by gene conversion to produce diversified humanized antibodies. The humanized antibodies obtained have no or minimal immunogenicity to humans and are appropriate for use in the therapeutic treatment of human subjects. WO 02/12437 further describes novel nucleotide sequences from the 5′ and 3′ flanking regions of immunoglobulin heavy chain constant region segments of various non-human mammalians, such as chickens, cows, sheep, and rabbits. Recombinant vectors in which human immunoglobulin heavy chain gene segments are flanked by sequences homologous to such 5′ and 3′ sequences are shown to be useful for replacing an immunoglobulin heavy chain gene segment of a non-human animal with the corresponding human immunoglobulin heavy chain gene segment.

While the methods of the invention are suitable for the introduction of immunoglobulin loci in the genome of non-human recipient animals in general, in a particular embodiment, the recombinase-mediated introduction of human or humanized immunoglobulin loci targets animals creating antibody diversity essentially by gene conversion.

Generally, all vertebrates start the creation of the primary antibody repertoire by recombining V, D, and J gene segments. In mice and humans this step results in considerable diversity as hundreds of VDJ genes are randomly recombined and genes are imprecisely joined together. In addition, in mice and humans rearrangement of V, D, and J elements occurs throughout life, resulting in the constant renewal of the primary antibody repertoire through the generation of new antibody producing B cells. However, in most other vertebrates, including chicken, rabbits, cows and sheep, this first step of VDJ recombination does not lead to significant diversity because only a limited number of genes are employed. For example, it is well established that in rabbit and chicken, VDJ rearrangement is very limited (almost 90% of immunoglobulin is generated with the 3′ proximal VH1 element). In addition, rearrangement of V, D and J elements stops around the time of birth, resulting in a very limited, fixed primary antibody repertoire, because no new antibody producing B-cells can be generated. To enhance diversity of the primary antibody repertoire such animals rely on a second step to modify antigen-binding regions through templated (gene conversion) and non-templated (hypermutation) mutational processes. Gene conversion creates broad diversity by modifying all three antigen-binding sites of the VDJ region.

The process of gene conversion transfers sequence information encoded (i.e. templated nucleotide substitutions) in upstream V genes to the rearranged exons. Gene-conversion events depend on a high sequence homology between donor and recipient V genes. In addition, certain structural features, in particular distance and orientation of immunoglobulin gene elements, are required for gene conversion to occur. A rearranged V gene undergoes about 10 gene-conversion events during B cell development, resulting in changes to each of the antigen-binding sites or complementarity-determining regions (CDRs). Insertion of sequences starts at sites in the recipient V gene where it shares extensive sequence homology with the donor V element and stops where sequence homology falls below a minimum threshold. For this reason, gene conversion selectively modifies CDR regions while leaving framework regions unaltered. The diversification of the primary antibody repertoire in gene converting animals, such as rabbits and chickens through gene conversion is significantly greater than the diversification of primary antibodies through rearrangement in rodents and humans because it allows the combination of fragments of several V gene segments.

As a consequence of gene conversion most high-affinity antibodies in gene converting animals contain a variable region which is substantially identical to a polypeptide sequence encoded by fragments of more than one V gene segments. This is a substantial difference between mice and humans (which do not use gene conversion for antibody diversification) where V regions of antibodies are always encoded by a single V element.

When the goal is introduction of one or more human or humanized immunoglobulin loci in the genome of a gene converting animal, the transgenic expression cassettes used are designed in such a way that antibody diversification by gene conversion can occur. Such humanized immunoglobulin loci retain the regulatory sequences of the exogenous Ig locus, and comprise two or more human Ig coding sequences, sequences flanked/separated by non-coding sequences from the non-human recipient animal. In other words, non-coding sequences of the recipient non-human animal are retained, and only coding sequences are replaced by coding sequences of a human Ig polypeptide. As a consequence, most (typically about 80% or more) of the novel humanized immunoglobulin locus consists of non-human sequences, and only a small part (typically about 20% or less) consists of sequences encoding human immunoglobulin polypeptides. In addition, V elements in such humanized Ig loci belong to families of V genes with at least 75% sequence identity and are configured for gene conversion. This design allows generation of a diversified human or humanized antibody repertoire by gene conversion in gene converting animals.

An Ig locus having the capacity to undergo gene rearrangement and gene conversion is also referred to as a “functional” Ig locus and the antibodies with a diversity generated by a functional Ig locus are also referred to as “functional” antibodies or a “functional” repertoire of antibody molecules.

In a particular embodiment of the methods of the present invention, the expression cassettes disclosed in WO 02/12437 can be modified by the inclusion of a recombinase specific recognition sequence (recombination site). It is, of course, equally possible to modify other transgenic expression constructs, carrying one or more human or humanized immunoglobulin loci, by the addition of a recombination site by methods well known in the art of genetic engineering.

Site-specific recombinases are enzymatically active proteins that catalyze a reciprocal double-stranded DNA exchange between two DNA segments. Such recombinases recognize specific sequences in both partners of the exchange and may function as sole proteins, or may require the presence of accessory factors for function. Site-specific recombinases are typically but not exclusively prokaryotic, e.g. bacterial proteins. The two largest families of site-specific recombinases in bacteria are λ integrase-like enzymes and the resolvase/invertases. Members of the two families significantly differ in their amino acid sequences, and in their mechanisms of catalysis. Recombination by members of the λ integrase family involves the formation and resolution of a Holliday junction intermediate during which the DNA is transiently attached to the enzyme through a phosphotyrosine linkage. The resolvase/invertase family of enzymes act via a concerted, four-strand staggered break and rejoining mechanism during which a phosphoserine linkage is formed between the enzyme and the DNA.

Site-specific recombinases are well known in the art, along with their recognition sequences. Thus, for example, the genome of the broad host range Streptomyces temperate phage, ΦC31 is known to integrate into the host chromosome with the aid of an enzyme that is a member of the resolvase/invertase family of site-specific recombinases. For further details see, e.g. Thorpe and Smith, Proc. Natl. Acad. Sci. USA, 95(10):5505-5510 (1998). The phage C31 integrase, has been shown to mediate efficient integration in the human cell environment at attB and attP phage attachment sites on extrachromosomal vectors. Other known and frequently used recombinases include Cre and FLP (see, e.g. Bouhassira et al., Blood 88 (Suppl. 1), 190a (1996); Bouhassira et al., Blood 90:3332-3344 (1997); Seibler & Bode, Biochemistry 36:1740-1747 (1997); Seibler et al., Biochemistry 37:6229-6234 (1998); Bethke & Sauer, Nucl. Acids Res. 25:2828-2834 (1997)). The target of the Cre recombinase is a 34-bp sequence that consists of two inverted 13-bp Cre-binding sites separated by an eight base spacer within which the recombination occurs (Hoess & Abremski, Proc. Natl. Acad. Sci. USA 81:1026-1029 (1984)).

According to the present invention, the recombinase may be introduced into the recipient cell before, concurrently with, or after introducing the transgene construct comprising the one or more human or humanized immunoglobulin loci and a recombinase specific recognition sequence. As noted before, in one embodiment, the recombinase is introduced into the cell as a mRNA, e.g. by injection into male pronuclei with the aid of a micromanipulator, as described in Example 5. Alternatively, the recombinase may be introduced into the recipient cell by a recombinant expression cassette (e.g. plasmid) encoding the recombinase. Such plasmids are known in the art and are either commercially available or can be readily made. For example, the cloning of ΦC31 integrase into a variant of the commercially available expression plasmid, pcDNA3 is described in Example 4. Cre expression plasmids are also commercially available, and include, for example pBS 185 (CMV-CRE) (Clontech). In a further embodiment, the recombinase is introduced into the recipient cell as an enzymatically active protein.

The expression cassettes used in the methods of the present invention can be introduced into a recipient animal by standard transgenic methods, such as by pronuclear injection using standard procedures. Such methods are described in the Examples below.

Instead of random integration, the methods of the present invention result in site-specific integration of the transgene containing one or more human(ized) Ig loci into the genome of recipient cells (such as fertilized oocyte or developing embryos). Preferably, such cells are derived from animal strains with an impaired expression of endogenous immunoglobulin genes. The use of such animal strains permits preferential expression of immunoglobulin molecules from the human(ized) transgenic Ig locus. Examples for such animals include the Alicia and Basilea rabbit strains, as well as Agammaglobinemic chicken strain, as well as immunoglobulin knock-out mice. Alternatively, transgenic animals with human(ized) immunoglobulin transgenes or loci can be mated with animal strains with impaired expression of endogenous immunoglobulins. Offspring homozygous for an impaired endogenous Ig locus and a human(ized) transgenic Ig locus can be obtained.

Alternatively, a transgenic vector can be introduced into appropriate animal recipient cells such as embryonic stem cells or already differentiated somatic cells. Afterwards, cells in which the transgene has integrated into the animal genome and has replaced the corresponding endogenous Ig locus by homologous recombination can be selected by standard methods. See for example, Kuroiwa et al, Nature Genetics 2004, Jun. 6. The selected cells may then be fused with enucleated nuclear transfer unit cells, e.g. oocytes or embryonic stem cells, cells which are totipotent and capable of forming a functional neonate. Fusion is performed in accordance with conventional techniques which are well established. Enucleation of oocytes and nuclear transfer can also be performed by microsurgery using injection pipettes. (See, for example, Wakayama et al., Nature (1998) 394:369.) The resulting egg cells are then cultivated in an appropriate medium, and transferred into synchronized recipients for generating transgenic animals. Alternatively, the selected genetically modified cells can be injected into developing embryos which are subsequently developed into chimeric animals.

Further, according to the present invention, a transgenic animal capable of producing human(ized) immunoglobulins can also be made by introducing into a recipient cell or cells, one or more of the recombination vectors described herein above, one of which carries a human Ig gene segment, linked to 5′ and 3′ flanking sequences that are homologous to the flanking sequences of the endogenous Ig gene segment, then selecting cells in which the endogenous Ig gene segment is replaced by the human Ig gene segment by homologous recombination, and deriving an animal from the selected genetically modified recipient cell or cells. Cells appropriate for use as recipient cells in this approach include embryonic stem cells or already differentiated somatic cells. A recombination vector carrying a human Ig gene segment can be introduced into such recipient cells by any feasible means, e.g., transfection. Afterwards, cells in which the human Ig gene segment has replaced the corresponding endogenous Ig gene segment by homologous recombination, can be selected by standard methods. These genetically modified cells can serve as nuclei donor cells in a nuclear transfer procedure for cloning a transgenic animal. Alternatively, the selected genetically modified embryonic stem cells can be injected into developing embryos which can be subsequently developed into chimeric animals.

In a specific embodiment, the transgene constructs of the invention may be introduced into the transgenic animals during embryonic life by directly injecting the transgenes into the embryo or indirectly by injecting them into the pregnant mother or into the egg-laying hen.

Transgenic animals produced by any of the foregoing methods form another embodiment of the present invention. The transgenic animals have at least one, i.e., one or more, human(ized) Ig loci in the genome, from which a functional repertoire of human(ized) antibodies is produced.

Once a transgenic non-human animal capable of producing diversified humanized immunoglobulin molecules is made, humanized immunoglobulins and humanized antibody preparations against an antigen can be readily obtained by immunizing the animal with the antigen. A variety of antigens can be used to immunize a transgenic host animal. Such antigens include, without limitation, microorganisms, e.g. viruses and unicellular organisms (such as bacteria and fungi), alive, attenuated or dead, fragments of the microorganisms, or antigenic molecules isolated from the microorganisms.

Exemplary bacterial antigens for use in immunizing an animal include purified antigens from Staphylococcus aureus such as capsular polysaccharides type 5 and 8, recombinant versions of virulence factors such as alpha-toxin, adhesin binding proteins, collagen binding proteins, and fibronectin binding proteins. Exemplary bacterial antigens also include an attenuated version of S. aureus, Pseudomonas aeruginosa, enterococcus, enterobacter, and Klebsiella pneumoniae, or culture supernatant from these bacteria cells. Other bacterial antigens which can be used in immunization include purified lipopolysaccharide (LPS), capsular antigens, capsular polysaccharides and/or recombinant versions of the outer membrane proteins, fibronectin binding proteins, endotoxin, and exotoxin from Pseudomonas aeruginosa, enterococcus, enterobacter, and Klebsiella pneumoniae.

Exemplary antigens for the generation of antibodies against fungi include attenuated version of fungi or outer membrane proteins thereof, which fungi include, but are not limited to, Candida albicans, Candida parapsilosis, Candida tropicalis, and Cryptococcus neoformans.

Exemplary antigens for use in immunization in order to generate antibodies against viruses include the envelop proteins and attenuated versions of viruses which include, but are not limited to respiratory synctial virus (RSV) (particularly the F-Protein), Hepatitis C virus (HCV), Hepatits B virus (HBV), cytomegalovirus (CMV), EBV, and HSV.

Therapeutic antibodies can be generated for the treatment of cancer by immunizing transgenic animals with isolated tumor cells or tumor cell lines; tumor-associated antigens which include, but are not limited to, Her-2-neu antigen (antibodies against which are useful for the treatment of breast cancer); CD19, CD20, CD22 and CD53 antigens (antibodies against which are useful for the treatment of B cell lymphomas), (3) prostate specific membrane antigen (PMSA) (antibodies against which are useful for the treatment of prostate cancer), and 17-1A molecule (antibodies against which are useful for the treatment of colon cancer).

The antigens can be administered to a transgenic host animal in any convenient manner, with or without an adjuvant, and can be administered in accordance with a predetermined schedule.

After immunization, serum or milk from the immunized transgenic animals can be fractionated for the purification of pharmaceutical grade polyclonal antibodies specific for the antigen. In the case of transgenic birds, antibodies can also be made by fractionating egg yolks. A concentrated, purified immunoglobulin fraction may be obtained by chromatography (affinity, ionic exchange, gel filtration, etc.), selective precipitation with salts such as ammonium sulfate, organic solvents such as ethanol, or polymers such as polyethyleneglycol.

For making a monoclonal antibody, spleen cells are isolated from the immunized transgenic animal and used either in cell fusion with transformed cell lines for the production of hybridomas, or cDNAs encoding antibodies are cloned by standard molecular biology techniques and expressed in transfected cells. The procedures for making monoclonal antibodies are well established in the art. See, e.g., European Patent Application 0 583 980 A1 (“Method For Generating Monoclonal Antibodies From Rabbits”), U.S. Pat. No. 4,977,081 (“Stable Rabbit-Mouse Hybridomas And Secretion Products Thereof”), WO 97/16537 (“Stable Chicken B-cell Line And Method of Use Thereof”), and EP 0 491 057 B1 (“Hybridoma Which Produces Avian Specific Immunoglobulin G”), the disclosures of which are incorporated herein by reference. In vitro production of monoclonal antibodies from cloned cDNA molecules has been described by Andris-Widhopf et al., “Methods for the generation of chicken monoclonal antibody fragments by phage display”, J Immunol Methods 242:159 (2000), and by Burton, D. R., “Phage display”, Immunotechnology 1:87 (1995), the disclosures of which are incorporated herein by reference.

Cells derived from the transgenic animals of the present invention, such as B cells or cell lines established from a transgenic animal immunized against an antigen, are also part of the present invention.

In a further aspect of the present invention, methods are provided for treating a disease in a primate, in particular, a human subject, by administering a purified humanized antibody composition, preferably, a humanized polyclonal antibody composition, desirable for treating such disease.

In another aspect of the present invention, purified monoclonal or polyclonal antibodies are admixed with an appropriate pharmaceutical carrier suitable for administration in primates especially humans, to provide pharmaceutical compositions. Pharmaceutically acceptable carriers which can be employed in the present pharmaceutical compositions can be any and all solvents, dispersion media, isotonic agents and the like. Except insofar as any conventional media, agent, diluent or carrier is detrimental to the recipient or to the therapeutic effectiveness of the antibodies contained therein, its use in the pharmaceutical compositions of the present invention is appropriate. The carrier can be liquid, semi-solid, e.g. pastes, or solid carriers. Examples of carriers include oils, water, saline solutions, alcohol, sugar, gel, lipids, liposomes, resins, porous matrices, binders, fillers, coatings, preservatives and the like, or combinations thereof.

The humanized polyclonal antibody compositions used for administration are generally characterized by containing a polyclonal antibody population, having immunoglobulin concentrations from 0.1 to 100 mg/ml, more usually from 1 to 10 mg/ml. The antibody composition may contain immunoglobulins of various isotypes. Alternatively, the antibody composition may contain antibodies of only one isotype, or a number of selected isotypes.

In most instances the antibody composition consists of unmodified immunoglobulins, i.e., humanized antibodies prepared from the animal without additional modification, e.g., by chemicals or enzymes. Alternatively, the immunoglobulin fraction may be subject to treatment such as enzymatic digestion (e.g. with pepsin, papain, plasmin, glycosidases, nucleases, etc.), heating, etc, and/or further fractionated.

The antibody compositions generally are administered into the vascular system, conveniently intravenously by injection or infusion via a catheter implanted into an appropriate vein. The antibody composition is administered at an appropriate rate, generally ranging from about 10 minutes to about 24 hours, more commonly from about 30 minutes to about 6 hours, in accordance with the rate at which the liquid can be accepted by the patient. Administration of the effective dosage may occur in a single infusion or in a series of infusions. Repeated infusions may be administered once a day, once a week once a month, or once every three months, depending on the half-life of the antibody preparation and the clinical indication. For applications on epithelial surfaces the antibody compositions are applied to the surface in need of treatment in an amount sufficient to provide the intended end result, and can be repeated as needed. In addition, antibodies can, for example, be administered as an intramuscular bolus injection, which may, but does not have to, be followed by continuous administration, e.g. by infusion.

The antibody compositions can be used to bind and neutralize antigenic entities in human body tissues that cause disease or that elicit undesired or abnormal immune responses. An “antigenic entity” is herein defined to encompass any soluble or cell-surface bound molecules including proteins, as well as cells or infectious disease-causing organisms or agents that are at least capable of binding to an antibody and preferably are also capable of stimulating an immune response.

Administration of an antibody composition against an infectious agent as a monotherapy or in combination with chemotherapy results in elimination of infectious particles. A single administration of antibodies decreases the number of infectious particles generally 10 to 100 fold, more commonly more than 1000-fold. Similarly, antibody therapy in patients with a malignant disease employed as a monotherapy or in combination with chemotherapy reduces the number of malignant cells generally 10 to 100 fold, or more than 1000-fold. Therapy may be repeated over an extended amount of time to assure the complete elimination of infectious particles, malignant cells, etc. In some instances, therapy with antibody preparations will be continued for extended periods of time in the absence of detectable amounts of infectious particles or undesirable cells. Similarly, the use of antibody therapy for the modulation of immune responses may consist of single or multiple administrations of therapeutic antibodies. Therapy may be continued for extended periods of time in the absence of any disease symptoms.

The subject treatment may be employed in conjunction with chemotherapy at dosages sufficient to inhibit infectious disease or malignancies. In autoimmune disease patients or transplant recipients, antibody therapy may be employed in conjunction with immunosuppressive therapy at dosages sufficient to inhibit immune reactions.

Further details of the invention will be apparent from the following non-limiting examples.

### EXAMPLE 1

Construction of a Humanized Rabbit Immunoglobulin Heavy Chain Locus Using Synthetic Fragments

BAC and fosmid clones containing rabbit immunoglobulin heavy chain locus sequences were isolated from genomic DNA libraries using probes specific for the constant, variable, and joining gene segments or the 3′ enhancer region. Isolated BACs 27N5 (GenBank Acc. No. AY386696), 219D23 (GenBank Acc. No. AY386695), 225P18 (GenBank Acc. No. AY386697), 38A2 (GenBank Acc. No. AY386694) and fosmid Fos15B (GenBank Acc. No. AY3866968) were sequenced (Ros et al., Gene 330, 49-59 (2004)). Selected immunoglobulin coding sequences (Cμ, Cγ, J_{H}) were exchanged with corresponding human counterparts by homologous recombination in E. Coli by ET cloning (E-Chiang Lee et al., Genomics 73, 56-65 (2001); Daiguan Yu et al., PNAS 97, 5978-5983 (2000); Muyrers et al., Nucleic Acids Research 27, 1555-1557 (1999); Zhang et al., Nature Biotechnology 18, 1314-1317 (2000)).

Four fragments denoted Unit 1, Unit 2, Unit 3, and Unit 4 with human V sequences and rabbit spacers were chemically synthesized. Each fragment was flanked 5′ by an AscI restriction endonuclease recognition sequence, 3′ by a lox71 Cre recombinase recognition sequence followed by Fse I and MluI restriction enzyme recognition sequences. Unit 2 consisted of human V_{H}3-49, V_{H}3-11, V_{H}3-7 and V_{H}3-15 variable genes separated by rabbit spacers I29-30, I3-4, I2-3 and the 3′ half of I1-2 (I1-2B). Unit 3 consisted of human V_{H}3-48, V_{H}3-43 and V_{H}3-64 separated by rabbit spacers I1-2A (5′ half of I1-2), I7-8, I6-7 and the 3′ half of I4-5 (I4-5B). Unit 4 consisted of human V_{H}3-74, V_{H}3-30, and V_{H}³-9 separated by the rabbit spacer sequences I4-5B, I26-27, I11-12 and I17-18.

In addition, Unit 4 had an Flp recombinase recognition target (FRT) sequence, followed by a SglfI restriction endonuclease recognition sequence preceding the already mentioned Asc I site.

Unit 1 had the human V_{H}3-23 gene 5′ flanked by the rabbit spacer I1-2, a lox66 Cre recombinase target sequence and an AscI endonuclease recognition sequence, and was 3′ flanked by IV-C (5′ half) rabbit spacer sequence followed by a MluI endonuclease recognition sequence.

A gentamycin selection cassette was PCR-amplified, using primers SEQ ID NOs 1 and 2 (Table 1) containing AscI and FseI sites and ligated into a pGEM vector with a modified cloning site including AscI, FseI, and MluI endonuclease recognition sites (pGEM.Genta modified by PCR using SEQ ID NOs 3 and 4, Table 1).

Units 2, 3 and 4 were cloned into pGEM.Genta (Promega) vectors.

Unit 1 was sub-cloned into a customized pBELOBAC 11 (NEB) vector linearized with Hind III, and PCR-amplified. The forward primer (SEQ ID NO: 5, Table 1) had restriction sites for HindIII, PacI and AatII, and the reverse primer (SEQ ID NO: 6) had restriction sites for Bam HI, MluI and AscI. The primers were designed in such a way that the pBELOBAC11 Chloramphenicol selection cassette was deleted. Furthermore, a Neomycin selection cassette was PCR-amplified with primers SEQ ID NOs: 7 and 8 (Table 1) carrying Bam HI and Hind III restriction sites, and ligated to the modified pBELOBAC 11 vector (pBB11.1).

Units 1-4 were assembled by cre-mediated recombination as described (Mejia et al, Genomics 70(2): 165-70 (2000)). First, Unit 2 was cloned into the customized pgem.Genta vector, digested with Fse I and subsequently recircularized by ligation. This vectorless construct was transformed into E. coli containing pBB11.l.Uniti and p706-Cre plasmid. Following recombination of Unit 2 with pBB 11.1.Unit 1, positive clones (Unit 1/2) were selected on kanamycin and gentamycin containing media. Clones were characterized by restriction analyses using various enzymes.

For recombination of Unit 3, the Unit1/2 insert was excised by double digestion with AscI and PacI, and cloned into pBELOBAC11 with a modified linker (pBB11.2: modified by PCR using primers SEQ ID NOs 9 and 10, Table 1).

pBELOBAC11 was linearized with HindIII and PCR-amplified with a forward primer encoding Pad and AatII endonuclease recognition sites and a reverse primer encoding MluI and NotI endonuclease recognition sites and a lox66 Cre recombinase target site. For ligation with Unit 1/2 the pBB 11.2 vector was opened with MluI and PacI.

pGEM.Genta.Unit3 was converted into a circular vectorless construct as described for pGEM.Genta.Unit2 and connected with pBB11.2.Unit1/2 by in vivo Cre mediated recombination. Subsequently, the resulting construct pBB 11.2.Unit1/2/3 was prepared for Cre mediated recombination with Unit 4 by replacing the wild type loxp site with a lox66 target site by ET-cloning (Muyrers et al., Nucleic Acids Research 27, 1555-1557 (1999); Muyrers et al Trends Biochem. Sci. 26(5):325-31 (2001)). A chloramphenicol selection cassette was amplified by PCR with primers (SEQ ID NOs 11 and 12, Table 1) containing 50bp sequences homologous to the BAC target sequence. The reverse primer included a lox66 site. The gel-purified PCR product was transformed into cells carrying the target BAC as well as the pSC101 plasmid, required for homologous recombination. Positive clones were selected with chloramphenicol and confirmed by restriction analysis and sequencing. pGEM.Genta.Unit 4 was prepared for in vivo recombination as described above for Units 2 and 3 and transformed into cells carrying the receptor BAC, as well as the p706-Cre plasmid. Positive clones pBB11.2.Unit1/2/3/4 were selected with gentamycin and confirmed by restriction analysis.

pBB11.2.Unit1/2/3/4 was further modified by ET-cloning to generate a lox 71 target site. Subsequently, pBB11.2.Unit1/2/3/4 was connected to fragments from BACs 219D23, 27N5 and Fos15B. The final construct FHHC (FIG. 1) was used for the generation of transgenic animals.

TABLE 1

| | | |
|---|---|---|
| ID | Region | Sequence |

| | | |
|---|---|---|
| 1 | Genta | 5′CCAGGCCGGCCTGGAGTTGTAGATCCTCTACG3′ |
| 2 | Genta | 5′CCAGGCGCGCCAAGATGCGTGATCTGATCC3′ |
| 3 | Linker | 5′GGCCGCGGCCGGCCATCGATGGCGCGCCTTCGAAACGCGTA3′ |
| 4 | Linker | 5′AGCTTACGCGTTTCGAAGGCGCGCCATCGATGGCCGGCCGC3′ |
| 5 | pBB11.1 | 5′ATTCCCAAGCTTTTAATTAAGACGTCAGCTTCCTTAGCTCCTG3′ |
| 6 | pBB11.1 | 5′ATTCGCGGATCCACGCGTTTCGTTCCCAAAGGCGCGCCTAGCGATGA |
| | | GCTCGGAC3′ |
| 7 | Neo | 5′GCAGGCATGCAAAGCTTATTACACCAGTGTCAGTAAGCG3′ |
| 8 | Neo | 5′GGTACCCGGGGATCCTCAGAAGAACTCGTCAAGAAGGCG3′ |
| 9 | pBB11.2 | 5′AAATTCCCTTAATTAAGACGTCAGCTTCCTTAGCTCCTG3′ |
| 10 | pBB11.2 | 5′GAAACCGGGGACGCGTTACCGTTCGTATAATGTATGCTATACGAAGT |
| | | TATGCGGCCGCTAGCGATGAGCTCGGAC3′ |
| 11 | CA | 5′TTCTCTGTTTTTGTCCGTGGAATGAACAATGGAAGTCCGAGCTCATC |
| | | GCTAAGGGCACCAATAACTGC3′ |
| 12 | CA | 5′CACAGGAGAGAAACAGGACCTAGAGGATGAGGAAGTCCCTGTAGGCT |
| | | TCCTACCGTTCGTATAATGTATGCTATACGAAGTTATTACCTGTGACGGAAG |
| | | ATC-3′ |
| 13 | Cκ Km3 | 5′GATGTCCACTGGTACCTAAGCCTCGCCCTCTGTGCTTCTTCCCTCCT |
| | | CAGGAACTGTGGCTGCACCATCTGTCTTC3′ |
| 14 | Cκ Km3 | 5′GAGGCTGGGCCTCAGGGTCGCTGGCGGTGCCCTGGCAGGCGTCTCGC |
| | | TCTAACACTCTCCCCTGTTGAAGCTCTTTGTG3 |
| 15 | Linker | 5′CGGGATCCGCGCGTACGGAAGTTCCTATACCTTTTGAAGAATAGGAA |
| | | CTTCGGAATAGGAACTTCATTACACCAGTGTCAGTAAGCG3′ |
| 16 | Linker | 5′GGGAAGCTTCGCGCGATCGCCGCTTTCGCAAAGGCGCGCCTCAGAAG |
| | | AACTCGTCAAGAAGGCG3′ |
| 17 | Genta | 5′GGCGGCCGCCTGGCCGTCGACATTTAGGTGACACTATAGAAGGATCC |
| | | GCGTGGAGTTGTAGATCCTCTACG3′ |
| 18 | Genta | 5′AACTCAGTAAGGAAAAGGACTGGGAAAGTGCACTTACATTTGATCTC |
| | | CAGGCGCGCCAAGATGCGTGATCTGATCC3′ |
| 19 | Neo | 5′GGACCAGTTTACAATCCCACCTGCCATCTAAGAAAGCTGGTCTCATC |
| | | GTGGTGCCAGGGCGTGCCCTTGGGCTGGGGGCGCGGAAGTTCCTATTCCGAA |
| | | GTTCCTATTCTTCAAAAGGTATAGGAACTTCCGTACGATTACACCAGTGTCA |
| | | GTAAGCG3′ |
| 20 | Neo | 5′GGACTGATGGGAAAATAGAGGAGAAAATTGACCAGAGGAAGTGCAGA |
| | | TGGTCAGAAGAACTCGTCAAGAAGGCG3′ |
| 21 | rpsL-neo | 5′CATACACAGCCATACATACGCGTGTGGCCGCTCTGCCTCTCTCTTGC |
| | Cλ | AGGTATTACACCAGTGTCAGTAAGCG3′ |
| 22 | rpsL-neo | 5′ATCAGGGTGACCCCTACGTTACACTCCTGTCACCAAGGAGTGGGAGG |
| | Cλ | GACTTCAGAAGAACTCGTCAAGAAGGCG3′ |
| 23 | rpsL-neo | 5′GGGGCCGTCACTGATTGCCGTFLTTCTCCCCTCTCTCCTCTCCCTCT |
| | VλJλ | CCAGATTACACCAGTGTCAGTAAGCG3′ |
| 24 | rpsL-neo | 5′CACAATTTCACGATGGGGGAAGAAAGACCGAGACGAGGTCAGCGACT |
| | VλJλ | CACTCAGAAGAACTCGTCAAGAAGGCG3′ |
| 25 | Kana | 5′TGTGTGAAATTGTTATCCGCTCACAATTCCACACAACATACGAGCCG |
| | | GGTGCCAGGGCGTGCCCTTGGGCTCCCCGGGCGCGTACTGGTTGGTCGACAC |
| | | TAGTATTACC′ |
| 26 | Kana | 5′CAATACGCAAACCGCGTCTCCCCGCGCGTTGGCCGATTCATTAATGG |
| | | AGCGGCGCGC CTAGGTGGACCAGTTGGTGATTTTG3′ |

### EXAMPLE 2

Construction of a Humanized Rabbit Light Chain Locus Containing Multiple Human Vκ Elements, Chicken Spacer Elements and a Rearranged Human VJ

Screening of a rabbit genomic BAC libraries resulted in the identification of three BACs 215M22, 179L1, and 19602 (GenBank Accession NOs: AY495826, AY495827, and AY495828, respectively) containing rabbit light chain K1 gene segments. Rabbit Cκ1 was exchanged with human Cκ allotype Km3 by ET cloning as described (E-Chiang Lee et al., Genomics 73, 56-65 (2001); Daiguan Yu et al., PNAS 97, 5978-5983 (2000); Muyrers et al., Nucleic Acids Research 27, 1555-1557 (1999); Zhang et al., Nature Biotechnology 18, 1314-1317 (2000)) Human Cκ (allotype Km3) was amplified by PCR with primers (SEQ ID Nos: 13 and 14, Table 1) containing 50bp sequences homologous to target sequences. Homology arms were designed based on the published sequence of rabbit germline kappa (b5; GenBank Accession No. K01363) and matched the intron-exon boundary of Cκ. The exchange of rabbit Cκ against the human Cκ in BAC 179L1 was verified by sequencing.

Two DNA fragments, Unit1 containing 17 human V pseudogenes and 18 chicken spacer sequences and Unit 2 containing one functional rearranged human kappa VJ gene with leader, 11 human V pseudogenes, 12 chicken spacer sequences and intron1 and parts of intron 2 were synthesized chemically and cloned into vector pBR322.

Units 1 and 2 were digested with the restriction enzymes NgoMIV and AsiSI or NgoMIV and AscI respectively and ligated into pBELOBAC11 with a modified linker by three fragment ligation. The modified linker contained a BsiWI restriction site, a FRT5-site, a rpsL-Neo-cassette, a AscI site and a AsiSI-site. The linker fragment was amplified with high fidelity polymerase (Roche), primers CE_{—}1_{—}001_{—}012904 (SEQ ID NO 15, Table 1) and CE_{—}1_on005_{—}013004 (SEQ ID NO 16, Table 1) and plasmid pRpsL-Neo (Genebridges, Germany) as template. Subsequently, the amplified product was ligated into Bam-HI and HindHi sites of pBELOBAC11. For ligation with Units 1 and 2, the modified pBELOBAC11 was opened with AsiSI and AscI. Positive clones (pBELOBAC11 Unit1/2) were checked by restriction enzyme digests.

BAC 179L1 (GenBank Acc. No. AY495827) was modified by insertion of two modified selection cassettes by ET cloning. Cassette 1 was a gentamycin resistance gene amplified with primers (SEQ ID Nos 17 and 18, Table 1) containing 50bp sequences homologous to BAC 179L1 and an AscI site in the reverse primer. Cassette 2 was a rpsl-Neo selection cassette amplified with primers (SEQ ID Nos 19 and 20, Table 1) containing 50bp sequences homologous to BAC 179L1 and an attB site, a FRT5 site and a BsiWI site in the forward primer.

The purified PCR products were transformed into E. coli cells carrying the BAC and plasmid pSC101 necessary for homologous recombination. After homologous recombination successful modification of the BAC was confirmed by restriction digest analyses, Southern Blot and sequencing.

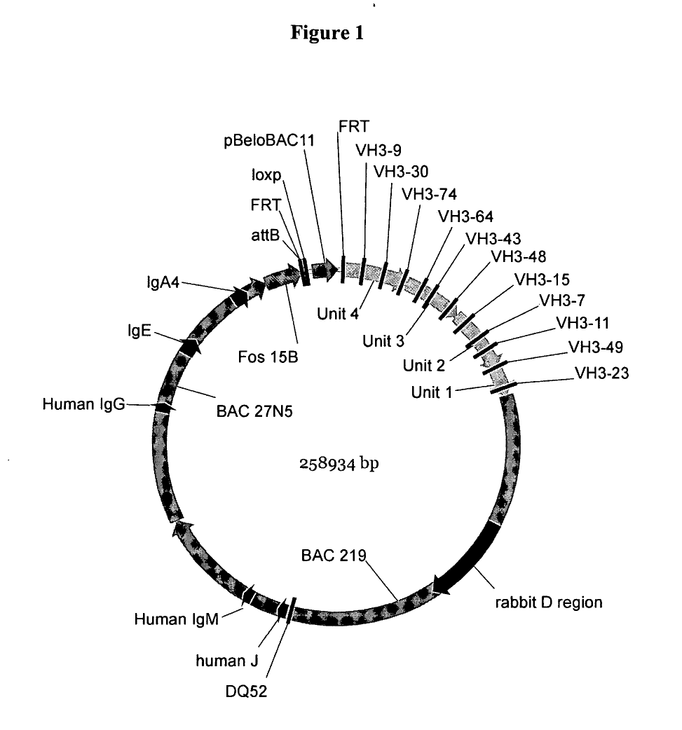

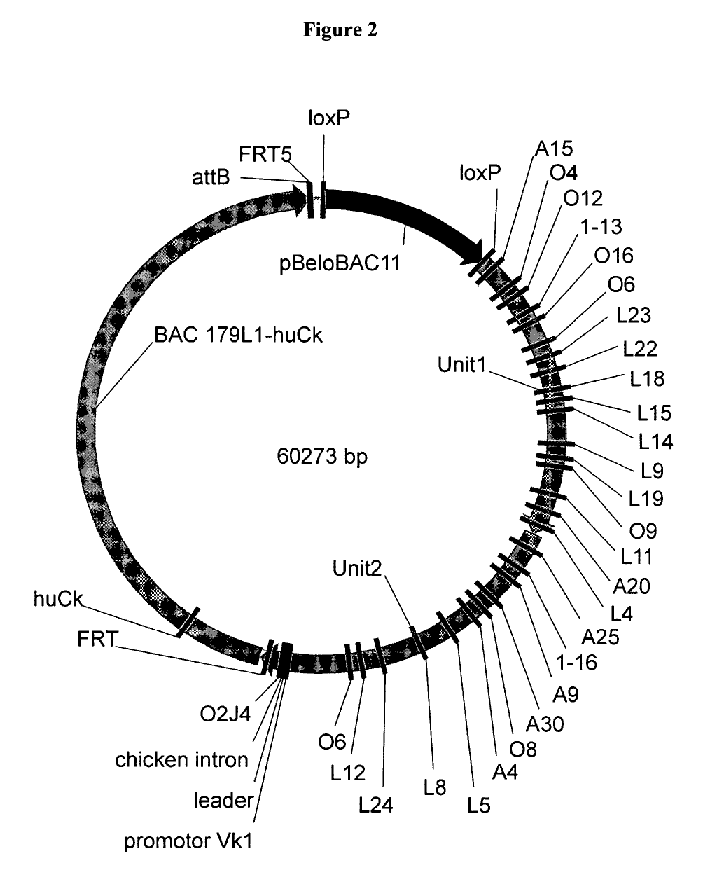

Modified BAC 179L1 was cut with the restriction enzymes AscI and BsiWI. The fragment containing the human Cκ was purified and ligated with pBELOBAC11 Unit1/2 opened with the same restriction enzymes. Positive clones were checked by restriction enzyme digests. The final construct (FIG. 2) was used for the generation of transgenic animals.

### EXAMPLE 3

Construction of a Humanized Chicken Lambda Light Chain Locus

A genomic BAC library derived from a jungle fowl chicken was screened with radiolabeled probes specific for chicken light chain Cλ and chicken Vpsi25 (the V gene segment at the very 5′ end of the light chain locus). A BAC clone containing the entire lambda light chain locus was identified. Chicken Cλ was replaced with human Cλ2 by homologous recombination in E.coli using the pET system (Zhang et al., Nat. Biotechnol. 18(12):1314-7, 2000) as follows.

A first DNA fragment containing a rpsL-neo selection/counterselection cassette was PCR amplified with specific primers (SEQ ID Nos: 21 and 22, Table 1). The 5′ primer included 50 bp derived from the 5′ flanking region of the chicken light chain Cλ gene. The 3′ primer included about 50 bp derived from the 3′ flanking region of the chicken light chain Cλ gene.

A second DNA fragment was synthesized using overlapping oligonucleotides wherein the DNA fragment contained from 5′ to 3′, a sequence derived from the 5′ flanking region of the chicken light chain Cλ gene, the human Cλ2 gene, and a sequence derived from the 3′ flanking region of the chicken Cλ gene.

E. coli cells of the chicken light chain BAC clone were transformed with a recombination plasmid expressing the recE and recT functions under an inducible promotor. Cells transformed with the recombination plasmid were then transformed with the first DNA fragment above and selected afterwards in media containing kanamycin. Clones resistant to kanamycin were identified and analyzed by restriction enzyme digest. Positive clones were further checked for streptomycin sensitivity conferred by rpsL.

In the second homologous recombination step, cells positive for the kanamycin selection cassette were transformed with the second DNA fragment above. Transformed cells were screened for streptomycin resistance and the loss of kanamycin resistance as indicative of the replacement of the rpsL-neo selection-/counterselection cassette by the human Cλ2 gene. The exchange was confirmed by restriction enzyme digest and/or sequence analysis.

The BAC clone containing the chicken light chain locus and the inserted human Cλ2 gene segment was further modified by inserting a rearranged VλJλ DNA fragment. The rearranged VλJλ DNA fragment encoded a human immunoglobulin variable domain polypeptide described by Kametani et al. (J. Biochem. 93 (2), 421-429, 1983) as IG LAMBDA CHAIN V-I REGION NIG-64 (P01702). The nucleotide sequence of the rearranged VλJλ fragment was altered in order to maximize the sequence homology with the chicken Vλ1 sequence published by McCormack et al. (Cell 56, 785-791, 1989). This rearranged VλJλ DNA sequence was more than 80% identical with known chicken light chain V genes. The rearranged VλJλ DNA fragment was linked to a 5′ flanking sequence and a 3′ flanking sequence. The 5′ flanking sequence was derived from 5′ of chicken Vλ1, and the 3′flanking sequence was derived from 3′ of chicken J. The DNA fragment was subsequently inserted into the chicken light chain locus in E. coli using the pET system. The insertion was performed in such a way that the region on the chicken light chain locus from the 5′ end of the chicken Vλ1 gene segment to the 3′ end of the chicken J region was replaced with the rearranged, synthetic VλJλ DNA fragment. Again, this insertion was accomplished by the replacement of the chicken V-J region with a PCR amplified marker gene (Primer SEQ ID NOs 23 and 24), followed by the replacement of the marker gene with the rearranged VλJλ DNA fragment. The modified BAC clone was amplified and purified using standard procedures.

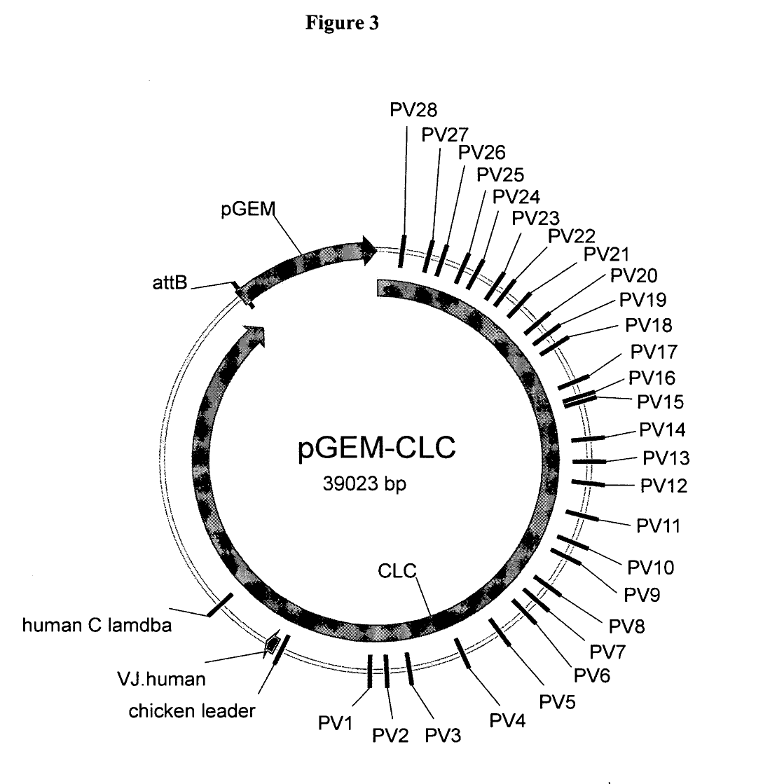

Subsequently, a 35 kb NotI fragment containing the entire humanized light chain locus was excised and cloned into pGEM13Zf(+) vector (=CLC-pGEM). A kanamycin-resistance cassette (neo) was PCR-amplified using primers attB40-neo.up (SEQ ID NO 25) and attB40-neo.do (SEQ ID NO 26). The primers contained 47bp and 50bp sequences derived from pGEM13Zf(+), respectively, and the up-primer additionally a 40bp-core region (CGGGTGCCAGGGCGTGCCCTTGGGCTCCCCGGGCGCGTAC) (SEQ ID NO: 28) of the attB site. The attB-neo-cassette was inserted into CLC-pGEM by ET cloning. The final construct (FIG. 3) was used for the generation of transgenic animals.

### EXAMPLE 4

Construction of a ΦC31-integrase Expression Plasmid

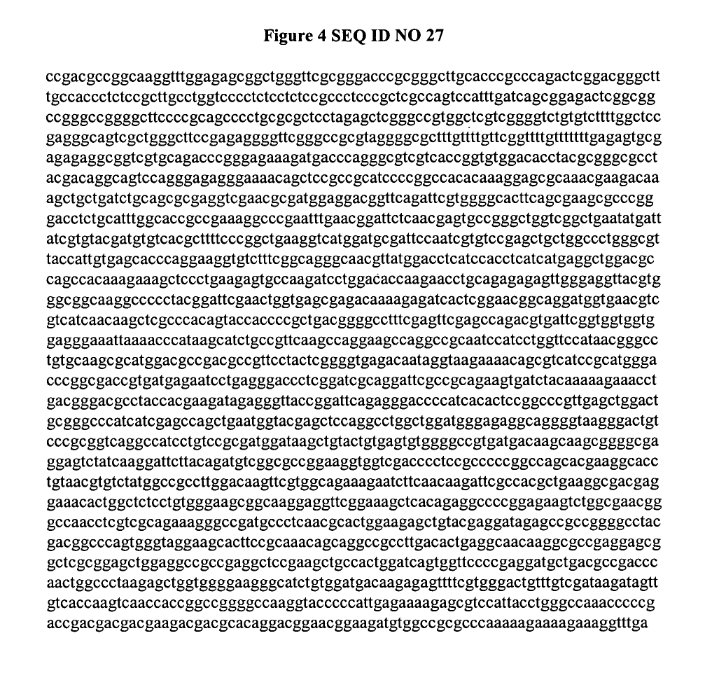

φC31 integrase gene was synthesized (SEQ ID NO: 27, FIG. 4) and cloned into pcDNA3.1. The sequence of the final product (pcDNA3.1-IRES-C31 -NLS) was confirmed.

### EXAMPLE 5

Transgenic Mice Expressing Humanized Immunoglobulins

Transgenic mice were generated by pronuclear injection using standard procedures. Briefly, female mice were superovulated using standard methods and mated with male mice. Pronuclear-stage zygotes were collected from oviduct and placed in M2 medium. Pronuclear DNA injection was performed with linearized CLC plasmid at 1.5 ng/μl or circular CLC plasmid (1.5ng/μl) combined with integrase mRNA (10 ng/μl). Integrase mRNA was generated by in vitro transcription of linearized pcDNA3.1-IRES-C31-NLS and subsequent RNA purification.

Nucleic acids were injected into male pronuclei with the aid of a pair of micromanipulators. Morphological surviving zygotes were transferred to the oviducts of pseudopregnant mice. Pseudopregnancy was induced by mating with sterile (vasectomized) males.

Linearized CLC plasmid DNA was injected in 726 pronuclei. Subsequently 425 embryos (59% of injected embryos) were transferred into 15 recipient females which gave birth to 26 offsprings. Six offspring were transgenic as shown by PCR corresponding to 0.8% of injected oocytes. Circular CLC+integrase mRNA was injected into 454 pronuclei. Subsequently 282 (62% of injected embryos) embryos were transferred into 10 recipients which gave birth to 50 live offsprings. Fourteen offsprings were transgenic corresponding to 3.1 % of injected oocytes.

Sandwich-type ELISAs detecting humanized lambda chains were performed using standard procedures. Briefly, microtiter plates were coated with capture antibody and incubated with diluted serum samples. Bound human immunoglobulin was detected using a secondary labeled antibody and peroxidase-streptavidin-conjugate (Sigma S-2438). In transgenic animals generated with linearized CLC expression levels in F1 progeny of the highest expressing transgenic line were 10.8±1.8 μg/ml. In animals generated with circular CLC+integrase mRNA expression levels were 20.4±18.1 μg/ml.

### EXAMPLE 5

Transgenic Rabbits Expressing Humanized Immunoglobulins

Transgenic rabbits were generated by pronuclear injection using standard procedures. Briefly, female rabbits were superovulated using standard methods and mated with male rabbits. Pronuclear-stage zygotes were collected from oviduct and placed in an appropriate medium such as Dulbecco's phosphate-buffered saline supplemented with 20% fetal bovine serum. Pronuclear DNA injection was performed with circular CLC plasmid (1.5 ng/μl) combined with integrase mRNA (10 ng/μl). Integrase m-RNA was generated by in vitro transcription of linearized pcDNA3.1-IRES-C31-NLS and subsequent RNA purification.

Nucleic acids were injected into male pronuclei with the aid of a pair of micromanipulators. Morphological surviving zygotes were transferred to the oviducts of pseudoprognant rabbits. Pseudopregnancy was induced by the injection of human chorionic gonadotrophin (hCG). Expression of human lambda light chain in founder animals was demonstrated by ELISA as described in Example 4.

### EXAMPLE 6

Modification of a Integrated Humanized Immunoglobulin Heavy Chain Locus Using Synthetic Fragments =P The FHHC construct from Example 1 is further modified by homologous recombination to insert a mutated loxp 2272 site at the 3′end of Unit1. Single copy transgenic animals are generated by C31-integrase mediated integration via the attB site. Positive animals are identified by PCR and tested for expression of the transgene. Offspring from transgenic founder animals are used for the introduction of additional DNA fragments into FHHC transgene. Transgenic female offspring are used as oozyte donors for microinjection, male offspring is used for sperm-mediated gene transfer or testis-mediated gene transfer.

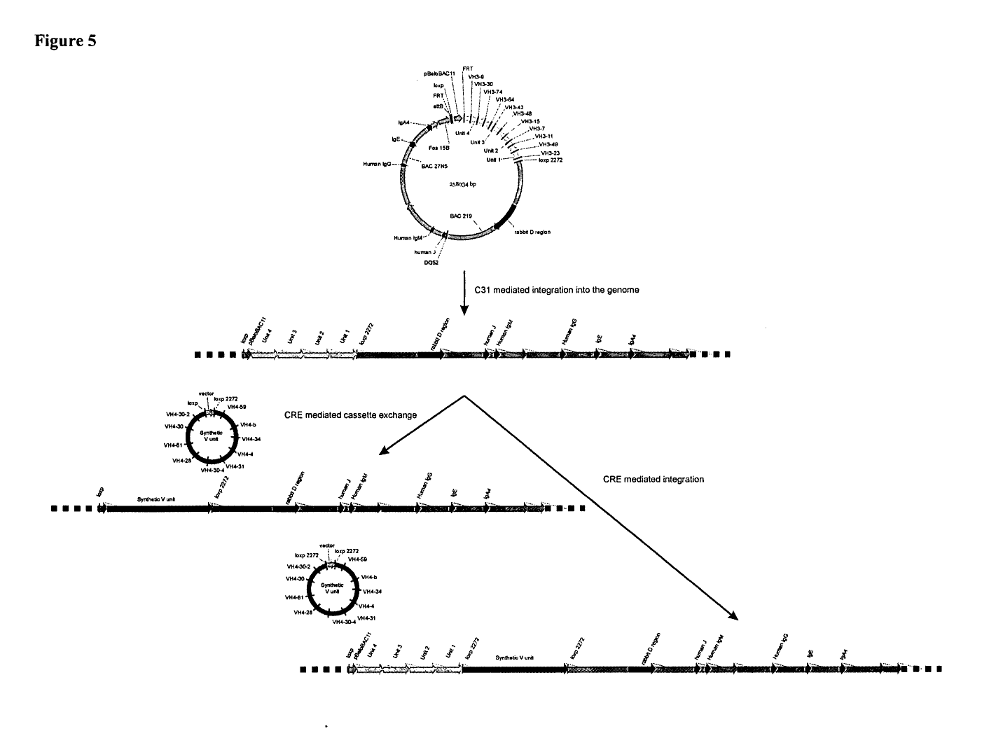

A synthetic DNA fragment with human VH4 gene elements separated by rabbit spacer sequences and flanked by a 5′loxP site and a 3′loxP2272 is used for further modification of the FHHC transgene. The synthetic fragment is coinjected with mRNA encoding Cre-recombinase. Dependent on the design of the injected DNA construct Cre mediates a cassette exchange or an insertion as outlined in FIG. 5. Founder animals with modified FHHC transgene are identified by PCR.

All references cited throughout the disclosure and all references cited therein are hereby expressly incorporated by reference.

While the invention is illustrated with reference to certain embodiments, it is not so limited. One skilled in the art will appreciate that certain variations are possible without diverting from the spirit of the invention. All such modification and variations are intended to be within the scope of the invention.

1. An isolated nucleic acid molecule comprising one or more human or humanized immunoglobulin loci and at least one recombination site.

2. The isolated nucleic acid molecule of claim 1, wherein said recombination site is the substrate for a recombinase selected from the group consisting of Cre-recombinase, Cre-like recombinase, FLP recombinase, and R recombinase.

3. The isolated nucleic acid molecule of claim 1, wherein said recombination site is the substrate for a transposase or a retrotransposase.

4. The isolated nucleic acid molecule of claim 2, which is circular.

5. The isolated nucleic acid molecule of claim 3, which is linear or circular.

6. A method for the site specific integration of a transgene comprising one or more human or humanized immunoglobulin loci in the genome of a eukaryotic cell, said method comprising:
introducing (i) a targeting construct comprising one or more human or humanized immunoglobulin loci and a first recombination site, and (ii) a site specific recombinase into a eukaryotic cell, wherein the genome of said cell comprises a second recombination site, maintaining the eukaryotic cell under conditions that allow recombination between the first and second recombination sites, wherein the recombination is mediated by the site specific recombinase and the result of the recombination is site specific integration of one or more human or humanized immunoglobulin loci in the genome of the eukaryotic cell.

7. The method of claim 6 wherein the recombinase is other than a transposase or a retrotransposase.

8. The method of claim 7, wherein said targeting construct is circular.

9. The method of claim 6, wherein the recombinase is a transposase or a retrotransposase.

10. The method of claim 9, wherein the targeting construct is linear or circular.

11. The method of claim 7, wherein the site-specific recombinase is a recombinase expressed by a phage.

12. The method of claim 11, wherein the phage is selected from the group consisting of (C31 integrase, TP901-1 and R4.

13. The method of claim 6, wherein the site-specific recombinase is a recombinase selected from the group consisting of Cre-recombinase, Cre-like recombinase, FLP recombinase, and R recombinase.

14. The method of claim 6, wherein the first and second recombination sites share at least 90% sequence identity.

15. The method of claim 6, wherein the first and second recombination sites share less than 90% sequence identity.

16. The method of claim 6, wherein the recombinase facilitates recombination between a bacterial genomic recombination site (attB) and a phage recombination site (attP).

17. The method of claim 16, wherein the first recombination site comprises an attB site, and the second recombination site comprises a pseudo-attP site.

18. The method of claim 16, wherein the first recombination site comprises an pseudo-attB site, and the second recombination site comprises an attP site.

19. The method of claim 17 or claim 18, wherein the recombinase is encoded by ΦC31 or phage R4 or TP901-1.

20. The method of claim 16, wherein the recombinase-facilitated recombination results in a site that is no longer a substrate for the recombinase.

21. The method of claim 16, wherein said recombinase is introduced into the cell as a polypeptide.

22. The method of claim 16, wherein said recombinase is introduced into the cell as a messenger RNA molecule encoding a recombinase polypeptide.

23. The method of claim 16, wherein said recombinase is introduced into the cells as an expression cassette encoding the recombinase polypeptide.

24. The method of claim 16, wherein said recombinase in introduced into the cell before introduction of said construct comprising one or more human or humanized immunogloblin loci.

25. The method of claim 16, wherein said recombinase in introduced into the cell concurrently with introduction of said construct comprising one or more human or humanized immunogloblin loci.

26. The method of claim 16, wherein said recombinase in introduced into the cell after introduction of said construct comprising one or more human or humanized immunogloblin loci.

27. A eukaryotic cell comprising one or more human or humanized immunoglobulin loci whose integration into the cellular genome was mediated by a recombinase.

28. A transgenic animal comprising at least one cell according to claim 27.


SEQUENCE LISTING (28 sequences)

SEQ ID NO: 1 - DNA, 32 bp, Artificial Sequence
  primer
ccaggccggcctggagttgtagatcctctacg

SEQ ID NO: 2 - DNA, 30 bp, Artificial Sequence
  primer
ccaggcgcgccaagatgcgtgatctgatcc

SEQ ID NO: 3 - DNA, 41 bp, Artificial Sequence
  primer
ggccgcggccggccatcgatggcgcgccttcgaaacgcgta

SEQ ID NO: 4 - DNA, 41 bp, Artificial Sequence
  primer
agcttacgcgtttcgaaggcgcgccatcgatggccggccgc

SEQ ID NO: 5 - DNA, 43 bp, Artificial Sequence
  primer
attcccaagcttttaattaagacgtcagcttccttagctcctg

SEQ ID NO: 6 - DNA, 55 bp, Artificial Sequence
  primer
attcgcggatccacgcgtttcgttcccaaaggcgcgcctagcgatgagctcggac

SEQ ID NO: 7 - DNA, 39 bp, Artificial Sequence
  primer
gcaggcatgcaaagcttattacaccagtgtcagtaagcg

SEQ ID NO: 8 - DNA, 39 bp, Artificial Sequence
  primer
ggtacccggggatcctcagaagaactcgtcaagaaggcg

SEQ ID NO: 9 - DNA, 39 bp, Artificial Sequence
  primer
aaattcccttaattaagacgtcagcttccttagctcctg

SEQ ID NO: 10 - DNA, 75 bp, Artificial Sequence
  primer
gaaaccggggacgcgttaccgttcgtataatgtatgctatacgaagttatgcggccgctagcgatgagctcggac

SEQ ID NO: 11 - DNA, 68 bp, Artificial Sequence
  primer
ttctctgtttttgtccgtggaatgaacaatggaagtccgagctcatcgctaagggcaccaataactgc

SEQ ID NO: 12 - DNA, 102 bp, Artificial Sequence
  primer
cacaggagagaaacaggacctagaggatgaggaagtccctgtaggcttcctaccgttcgtataatgtatgctatacgaagttattacctgtgacggaagatc

SEQ ID NO: 13 - DNA, 76 bp, Artificial Sequence
  primer
gatgtccactggtacctaagcctcgccctctgtgcttcttccctcctcaggaactgtggctgcaccatctgtcttc

SEQ ID NO: 14 - DNA, 79 bp, Artificial Sequence
  primer
gaggctgggcctcagggtcgctggcggtgccctggcaggcgtctcgctctaacactctcccctgttgaagctctttgtg

SEQ ID NO: 15 - DNA, 87 bp, Artificial Sequence
  primer
cgggatccgcgcgtacggaagttcctataccttttgaagaataggaacttcggaataggaacttcattacaccagtgtcagtaagcg

SEQ ID NO: 16 - DNA, 64 bp, Artificial Sequence
  primer
gggaagcttcgcgcgatcgccgctttcgcaaaggcgcgcctcagaagaactcgtcaagaaggcg

SEQ ID NO: 17 - DNA, 71 bp, Artificial Sequence
  primer
ggcggccgcctggccgtcgacatttaggtgacactatagaaggatccgcgtggagttgtagatcctctacg

SEQ ID NO: 18 - DNA, 76 bp, Artificial Sequence
  primer
aactcagtaaggaaaaggactgggaaagtgcacttacatttgatctccaggcgcgccaagatgcgtgatctgatcc

SEQ ID NO: 19 - DNA, 158 bp, Artificial Sequence
  primer
ggaccagtttacaatcccacctgccatctaagaaagctggtctcatcgtggtgccagggcgtgcccttgggctgggggcgcggaagttcctattccgaagttcctattcttcaaaaggtataggaacttccgtacgattacaccagtgtcagtaagcg

SEQ ID NO: 20 - DNA, 74 bp, Artificial Sequence
  primer
ggactgatgggaaaatagaggagaaaattgaccagaggaagtgcagatggtcagaagaactcgtcaagaaggcg

SEQ ID NO: 21 - DNA, 73 bp, Artificial Sequence
  primer
catacacagccatacatacgcgtgtggccgctctgcctctctcttgcaggtattacaccagtgtcagtaagcg

SEQ ID NO: 22 - DNA, 75 bp, Artificial Sequence
  primer
atcagggtgacccctacgttacactcctgtcaccaaggagtgggagggacttcagaagaactcgtcaagaaggcg

SEQ ID NO: 23 - DNA, 72 bp, Artificial Sequence
  primer
ggggccgtcactgattgccgttttctcccctctctcctctccctctccagattacaccagtgtcagtaagcg

SEQ ID NO: 24 - DNA, 74 bp, Artificial Sequence
  primer
cacaatttcacgatgggggaagaaagaccgagacgaggtcagcgactcactcagaagaactcgtcaagaaggcg

SEQ ID NO: 25 - DNA, 109 bp, Artificial Sequence
  primer
tgtgtgaaattgttatccgctcacaattccacacaacatacgagccgggtgccagggcgtgcccttgggctccccgggcgcgtactggttggtcgacactagtattacc

SEQ ID NO: 26 - DNA, 82 bp, Artificial Sequence
  primer
caatacgcaaaccgcctctccccgcgcgttggccgattcattaatgcagcggcgcgcctaggtggaccagttggtgattttg

SEQ ID NO: 27 - DNA, 2219 bp, Artificial Sequence
  C31 integrase gene
ccgacgccggcaaggtttggagagcggctgggttcgcgggacccgcgggcttgcacccgcccagactcggacgggctttgccaccctctccgcttgcctggtcccctctcctctccgccctcccgctcgccagtccatttgatcagcggagactcggcggccgggccggggcttccccgcagcccctgcgcgctcctagagctcgggccgtggctcgtcggggtctgtgtcttttggctccgagggcagtcgctgggcttccgagaggggttcgggccgcgtaggggcgctttgttttgttcggttttgtttttttgagagtgcgagagaggcggtcgtgcagacccgggagaaagatgacccagggcgtcgtcaccggtgtggacacctacgcgggcgcctacgacaggcagtccagggagagggaaaacagctccgccgcatccccggccacacaaaggagcgcaaacgaagacaaagctgctgatctgcagcgcgaggtcgaacgcgatggaggacggttcagattcgtggggcacttcagcgaagcgcccgggacctctgcatttggcaccgccgaaaggcccgaatttgaacggattctcaacgagtgccgggctggtcggctgaatatgattatcgtgtacgatgtgtcacgcttttcccggctgaaggtcatggatgcgattccaatcgtgtccgagctgctggccctgggcgttaccattgtgagcacccaggaaggtgtctttcggcagggcaacgttatggacctcatccacctcatcatgaggctggacgccagccacaaagaaagctccctgaagagtgccaagatcctggacaccaagaacctgcagagagagttgggaggttacgtgggcggcaaggccccctacggattcgaactggtgagcgagacaaaagagatcactcggaacggcaggatggtgaacgtcgtcatcaacaagctcgcccacagtaccaccccgctgacggggcctttcgagttcgagccagacgtgattcggtggtggtggagggaaattaaaacccataagcatctgccgttcaagccaggaagccaggccgcaatccatcctggttccataacgggcctgtgcaagcgcatggacgccgacgccgttcctactcggggtgagacaataggtaagaaaacagcgtcatccgcatgggacccggcgaccgtgatgagaatcctgagggaccctcggatcgcaggattcgccgcagaagtgatctacaaaaagaaacctgacgggacgcctaccacgaagatagagggttaccggattcagagggaccccatcacactccggcccgttgagctggactgcgggcccatcatcgagccagctgaatggtacgagctccaggcctggctggatgggagaggcaggggtaagggactgtcccgcggtcaggccatcctgtccgcgatggataagctgtactgtgagtgtggggccgtgatgacaagcaagcggggcgaggagtctatcaaggattcttacagatgtcggcgccggaaggtggtcgacccctccgcccccggccagcacgaaggcacctgtaacgtgtctatggccgccttggacaagttcgtggcagaaagaatcttcaacaagattcgccacgctgaaggcgacgaggaaacactggctctcctgtgggaagcggcaaggaggttcggaaagctcacagaggccccggagaagtctggcgaacgggccaacctcgtcgcagaaagggccgatgccctcaacgcactggaagagctgtacgaggatagagccgccggggcctacgacggcccagtgggtaggaagcacttccgcaaacagcaggccgccttgacactgaggcaacaaggcgccgaggagcggctcgcggagctggaggccgccgaggctccgaagctgccactggatcagtggttccccgaggatgctgacgccgacccaactggccctaagagctggtggggaagggcatctgtggatgacaagagagttttcgtgggactgtttgtcgataagatagttgtcaccaagtcaaccaccggccggggccaaggtacccccattgagaaaagagcgtccattacctgggccaaacccccgaccgacgacgacgaagacgacgcacaggacggaacggaagatgtggccgcgcccaaaaagaaaagaaaggtttga

SEQ ID NO: 28 - DNA, 40 bp, Artificial Sequence
  Core region of the attB site
cgggtgccagggcgtgcccttgggctccccgggcgcgtac